(19) United States
(12) Patent Application Publication
(10) Pub. No.: US 2001/0046491 A1
(43) Pub. Date: Nov. 29, 2001

(54) Tumor radiosensitization with mutant thymidine kinase in combination with a prodrug

(76) Inventor: Kristoffer C. Valerie, Midlothian, VA (US)

Correspondence Address: McGuireWoods, LLP, 1750 Tysons Boulevard., Suite 1800, Tysons Corner, McLean, VA, 22102-4215, US

(21) Appl. No.: 09/814,938

(22) Filed: Mar. 23, 2001

Related U.S. Application Data
(60) Provisional application No. 60/191,544, filed on Mar. 23, 2000.

(51) Int. Cl. A61K 48/00; A61K 31/522

(52) U.S. Cl. 424/93.21; 514/44; 514/262

(57) ABSTRACT

The present invention provides a method for radiosensitizing cancer cells and tumors by utilizing a high activity form of thymidine kinase (TK) and a prodrug. The inventions also provides a method for killing cancer cells and for treating cancerous tumors in a mammal by utilizing a high activity form of thymidine kinase and a prodrug, in combination with radiation. In preferred embodiments, the high activity form of TK is a mutant of herpes simplex virus (HSV) TK and the prodrug is acyclovir.

This application claims priority to U.S. provisional application Ser. No. 60/191,544 filed Mar. 23, 2000.

This invention was made in part using funds from grants from the National Cancer Institute/National Institutes of Health having grant number CA61945. The government may have certain rights in this invention.

### DESCRIPTION

### BACKGROUND OF THE INVENTION

1. Field of the Invention

The invention generally relates to the radiosensitization of cancer cells and tumors. In particular, the invention provides a method to more effectively sensitize cancer cells or tumors to radiation therapy using forms of thymidine kinase (TK) that demonstrate increased prodrug phosphorylation activity compared to wild type herpes simplex virus thymidine kinase (HSV-TK), in combination with a prodrug.

2. Background of the Invention

Radiation therapy is frequently prescribed for the treatment of various types of human cancers. However, this method has serious drawbacks in that the radiation, while killing cancer cells, is also toxic to normal tissues. This normal tissue toxicity leads to side effects that limit how much radiation can be delivered to a tumor, and, under certain circumstances, may even be life threatening. As a result, the dosage, frequency and location at which radiation therapy can be administered are limited. There is therefore much interest in developing ways to increase the radiosensitivity of cancer cells so that lower or less frequent doses of radiation can be administered, while still achieving the same (or an improved) level of killing of cancer cells and destroying tumors.

For example, it has been demonstrated that glioma cells can be sensitized to radiation both in vitro and in vivo after infection with an adenovirus expressing the thymidine kinase enzyme from herpes simplex virus (HSV-TK), followed by exposure to a suitable prodrug, for example, the halogenated pyrimidine bromodeoxycytidine (Brust et al, 2000), or the clinically used anti-herpetic drug acyclovir (ACV) (Kim et al., 1995; Kim et al., 1997). The most commonly used prodrug for HSV-TK therapy is ganciclovir (GCV). However, this drug has serious immuno- and myelo-suppressive effects which seriously limit its clinical usefulness.

Other antitumor treatments that utilize wild type thymidine kinase in combination with prodrugs are described in U.S. Pat. No. 5,985,266 to Link, Jr. et al. issued Nov. 16, 1999 and U.S. Pat. No. 6,066,624 to Woo et al., issued May 23, 2000.

The efficient clinical use of the HSV-TK/ACV combination for radiosensitization of tumors has thus far been limited because wild type HSV-TK cannot efficiently utilize the low (micromolar) concentrations of ACV that can be achieved in sera and the central nervous system (CNS). Unlike GCV, the maximum serum concentration of ACV that can be achieved is not dictated by toxicity (ACV is known to be relatively non-toxic) but rather by metabolism and the rate of clearance from the body.

Another difficulty that applies specifically to the treatment of brain tumors with drugs including HSV-TK and a suitable prodrug is to overcome the barrier for transporting drugs past the blood-brain-barrier (BBB). The BBB impedes molecules larger than 200 Da from passing from the bloodstream into the brain. However, small hydrophobic drugs such as ACV can traverse the BBB. That is the reason why this drug is used, for example, as the preferred treatment for herpes-associated encephalitis.

It would be a distinct advantage to have available more effective methods of radiosensitizing cancer cells and tumors that overcome these clinical and physical obstacles. In particular, it would be highly advantageous to have available a method of radiosensitizing cancer cells in the brain that utilizes a combination of TK and ACV since ACV is known to be relatively non-toxic.

### SUMMARY OF THE INVENTION

It is an object of this invention to provide a method of radiosensitizing cancer cells or tumors. The method comprises contacting the cancer cells or tumor with a vector encoding a form of thymidine kinase (TK) that displays increased activity with respect to phosphorylation of a nucleoside analog (prodrug), compared to wild type HSV-TK, in combination with a administration of a prodrug. Without being bound by theory, it is believed that the resulting phosphorylated prodrug (i.e. the active drug) is incorporated into DNA and inhibits DNA synthesis, thereby resulting in increased radio sensitization. In some embodiments of the present invention, the vector is an adenoviral vector. In a preferred embodiment of the present invention, the vector is AdCMV-TK75. In preferred embodiments of the present invention, the form of TK is a mutant of HSV-TK. In another preferred embodiment, the form of TK is the HSV-TK mutant HSV-TK75. In a preferred embodiment of the invention, the prodrug is acyclovir.

The instant invention provides a method of killing cancer cells and suppressing the growth of tumors. The method comprises contacting the cancer cells or tumor with a vector encoding a form of TK that displays increased activity with respect to phosphorylation of nucleoside analogs when compared to wild type HSV-TK, in combination with administration of a prodrug, and delivery of a dose of radiation sufficient to kill the cancer cells. In some embodiments of the method, the vector is an adenoviral vector. In a preferred embodiment of the method, the vector is AdCMV-TK75. In preferred embodiments of the present invention, the form of TK is a mutant of HSV-TK. In yet another preferred embodiment, the form of TK is the HSV-TK mutant HSV-TK75. In a preferred embodiment of the invention, the prodrug is acyclovir.

The present invention provides a method for the treatment of cancer in a mammal. The method comprises contacting the cancer with a vector encoding a form of TK that displays increased activity with respect to phosphorylation of nucleoside analogs when compared to wild type HSV-TK, in combination with administration of a prodrug, and delivery of radiation. In some embodiments of the method, the vector is an adenoviral vector. In a preferred embodiment of the method, the vector is AdCMV-TK75. In preferred embodiments of the present invention, the form of TK is a mutant of HSV-TK. In yet another preferred embodiment, the form of TK is the HSV-TK mutant HSV-TK75. In a preferred embodiment of the invention, the prodrug is acyclovir.

### ABBREVIATIONS

ACV: acyclovir

BBB: blood brain barrier

CMV: cytomegalovirus

GCV: ganciclovir

HSV: herpes simplex virus

HSV-TK: herpes simplex virus thymidine kinase

IR: ionizing radiation

MOI: multiplicity of infection

MTT: 3-(4,5-dimethylthiazol-2-yl)-2,5-dirnethyltetrazolium bromide

SER: sensitizer enhancement ratio

TK: thymidine kinase

TUNEL: terminal uridyl-nucleotide end labeling

### BRIEF DESCRIPTION OF THE DRAWINGS

FIG. 1. Western blot analysis of extracts from rat glioma RT2 cells infected with adenovirus expressing either wild type or mutant HSV-TK75. Rat RT2 cells were infected with either Adβgal, AdCMV-TK, or AdCMV-TK75 adenovirus. After 3 days, infected cells were prepared for sodium dodecyl sulfate-polyacrylamide gel electrophoresis analysis and Western blotting with rabbit anti-HSV-TK Ab. The 44-kDa HSV-TK protein is indicated by an arrow. The weak band seen in the lane with extract from Adβgal-infected cells is a cross-reactive protein of unknown identity.

FIG. 2. Mutant HSV-TK75 sensitizes rat RT2 glioma cells to GCV and ACV more efficiently than wild type HSV-TK. Rat RT2 cells were infected with AdCMV (empty vector), AdCMV-TK, or AdCMV-TK75. At 2 days postinfection, cells were trypsinized, serially diluted, seeded in microtiter plates, and exposed to various doses of ACV or GCV. After 5 days, the plates were processed for growth/toxicity by MTT assay. (Contessa et al., 1999). Values were normalized to values from cells that were infected but not treated with GCV or ACV.

FIG. 3. Radiation potentiates the killing of rat RT2 glioma cells exposed to low doses of ACV and infected with adenovirus expressing mutant HSV-TK75. Rat RT2 cells were left uninfected or infected with AdCMV (empty vector), AdCMV-TK, or AdCMV-TK75. At 2 days postinfection, cells were trypsinized, serially diluted in micro-titer plates, exposed to ACV at either 1 microM or 3 microM for 20 hours, and then irradiated with either 2 or 4 Gy. After 5 days, MTT assays in triplicate determined the effect on cell growth/toxicity. Values were normalized to the values obtained without any ACV treatment. The SEM is indicated by error bars.

FIG. 4. Radiation potentiates the apoptosis of rat RT2 glioma cells exposed to ACV and infected with adenovirus expressing mutant HSV-TK75. Rat RT2 cells were infected with AdCMV (empty vector), AdCMV-TK, or AdCMV-TK75. At 2 days postinfection, cells were exposed to 3 microM ACV for 20 hours and then irradiated with either 2 or 5 Gy. At 20 hours postirradiation, cells were examined for apoptosis by terminal deoxynucleotidyltransferase-mediated deoxyuridine triphosphate nick end labeling (TUNEL assay). A representative result is shown.

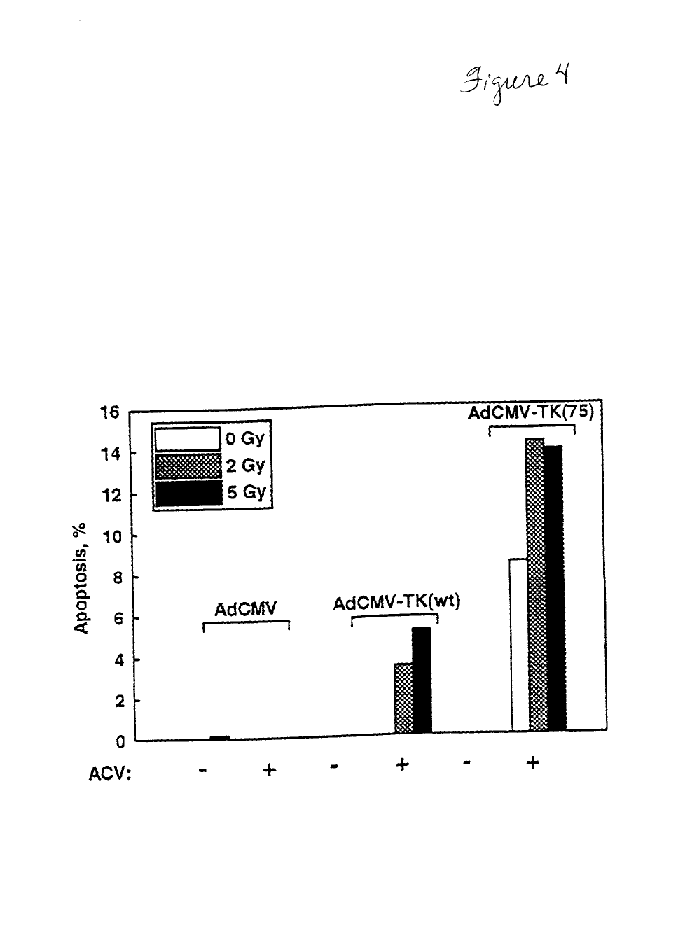

FIG. 5. Transduction of rat RT2 glioma cells with an adenovirus expressing wild type HSV-TK and exposure to acyclovir radiosensitize intracerebrally implanted RT2 gliomas in vivo. Kaplan-Meier survival plot of Fischer 344 rats (3 animals per group) intracerebrally implanted with RT2 cells (3×10⁴) transduced with either Adβgal or AdCMV-TK at an MOI of 10. Osmotic pumps were implanted immediately after implantation of transduced cells. Five days post-implantation, 7.25 Gy was delivered to the brain on three consecutive days (+IR). There were statistically significant differences (p<0.001) between the mean survival times of the four groups: ●, Adβgal(−IR); ▾, AdCMV-TK(−IR); ∘, Adβgal(+IR); ∇, AdCMV-TK(+IR). In pair-wise comparison, there was a statistically significant difference (**, p=0.02) between the mean survival times of animals treated with AdCMV-TK(−IR) and AdCMV-TK(+IR), and between Adβgal(−IR), and Adβgal(+IR) (*, p=0.025). To adjust for multiple comparison, Bonnferonicorrection corresponding to a p-value of 0.025 was considered.

FIG. 6. Treatment with mutant HSV-TK75 and acyclovir only marginally improves survival of rats compared to treatment with wild type HSV-TK. Kaplan-Meier survival plot of Fischer 344 rats (6 animals per group) intracerebrally implanted RT2 cells (3×10⁴) transduced with either Adβgal, AdCMV-TK, or AdCMV-TK75. Animals were treated the same way as described in the legend of FIG. 5, except that AdCMV-TK75 was also included in this experiment. RT2 cells were transduced at an MOI of 30. There were statistically significant differences (p=0.002) between the mean survival times of the three groups: ●, Adβgal; ∘, AdCMV-TK; and ▾ AdCMV-TK75. In pair-wise comparison, there was a statistically significant difference (*, p<0.001) between the mean survival times of Adβgal and AdCMV-TK. However, there was no statistically significant difference (**, p=0.19) between the mean survival times of animal implanted with AdCMV-TK- and AdCMV-TK75-transduced RT2 cells. To adjust for multiple comparison, Bonnferoni correction corresponding to p=0.025 was considered.

FIG. 7. Substantially improved radiosensitization of rat RT2 gliomas with mutant HSV-TK75 in combination with acyclovir. Kaplan-Meier survival plot of Fischer 344 rats intracerebrally implanted with RT2 cells (3×10⁴) transduced with Adβgal, AdCMV-TK, or AdCMV-TK75. Six animals each were implanted with cells transduced with one of the three viruses. Half the animals in each group were irradiated (+IR) and the other half was left unirradiated (−IR). There were statistically significant differences (p<0.001) between the mean survival times of the six groups: ●, Adβgal(−IR); ▾, AdCMV-TK(−IR); ▪, AdCMV-TK75(−IR); ∘, Adβgal(+IR); ∇, AdCMV-TK(+IR); □, AdCMV-TK75(+IR). The animals treated with AdCMV-TK75(+IR) were sacrificed on day 80 (*). In pair-wise comparison, there was a statistically significant difference (**, p=0.005) between the mean survival times of animals implanted with cells transduced with AdCMV-TK75 that did not receive any radiation, AdCMV-TK75(−IR), and animals that did, AdCMV-TK75(+IR). However, there was no statistically significant difference (p=0.07) between AdCMV-TK(−IR) and AdCMV-TK(+IR). There was also no statistically significant difference (p=0.20) between Adβgal(−IR) and Adβgal(+IR). There was also no statistically significant difference (p=0.03 vs. p=0.0125 and family-wise error rate=0.05) between AdCMV-TK(−IR) and AdCMV-TK75(−IR). To adjust for multiple comparison, Bonnferoni correction corresponding top=0.0125 was considered.

FIGS. 8A-F. Detection of tumor in brain sections of RT2-implanted rats. A Fischer 344 brain, which had received Adβgal-transduced RT2 cells, was collected and fixed in 10% formalin after the animal was determined moribund. The fixed brain was imbedded in paraffin and coronal sections were cut at a thickness of 5 mm and stained with hematoxylin and eosin. The tumor mass can easily be seen as a darker-stained area (arrow) at low power (0.5×) in 8A. At higher power (40×), a swirling cell pattern of darker-stained cells indicative of tumor cells, can be observed in 8B. At even higher power (100×/oil-immersion) individual tumor and infiltrating inflammatory cells can be seen invading the parenchyma (8C). Similarly, brain sections from an animal implanted with RT2 cells transduced with AdCMV-TK75 sacrificed on day 80 was stained with H&E (FIGS. 8D, 8E, 8F). A very small lesion can be detected in 8D (rectangle) and 8E. Dark-stained tumor cells are shown in 8F. The bars represents 1.5 mm.

FIG. 9. Colony-forming ability of U87 cells transduced with AdCMV-TK, AdCMV-TK75, or Adβgal and ACV followed by radiation. U87 cells were transduced with the appropriate virus at MOI=10, and two days later, trypsinized, diluted, and plated in 10-cm dishes. The cells were allowed to attach for several hours, then treated with 3 microM ACV. Twenty-four hours after addition of ACV, the dishes were irradiated (⁶⁰Co), and 24 h later the ACV removed, and the medium replaced with fresh media. The cells were then incubated for a further 12 days (14 days total since plating), stained with crystal violet, and colonies counted. In parallel, transduced cells were analyzed by Western blotting using anti-β-actin antibody (using chemiluminescence) and subsequently probed for HSV-TK (using CDP-Star). Equal expression of wild type and mutant HSV-TK was found (data not shown.) ♦=no virus; ●=Adβgal; ▪=AdCMV-TK; ▴=AdCMV-TK75.

FIGS. 10A and B. Radiosensitization of U87MG xenografts grown in the flank of nu/nu mice transduced with AdCMV-TK75 or Adβgal and administration of ACV. U87MG xenografts grown in nu/nu mice were infused with 2.2×10⁹ pfu/tumor of AdCMV-TK75 or Adβgal and treated with acyclovir (100 mg/kg BID) for 5 days and then treated with radiation on 3 consecutive days (3×10 Gy) following ACV administration (10A). ●=AdCMV-TK75 without radiation; ∘=AdCMV-TK75 plus radiation; ▪=Adβgal without radiation; □=Adβgal plus radiation. U87MG xenografts were infused with AdCMV-TK75 as above and treated with ACV (100 mg/kg BID for 5 days) concurrently with radiation (3×10 Gy) (10B). Δ=AdCMV-TK75 without radiation; ▴=AdCMV-TK75 plus radiation.

### DETAILED DESCRIPTION OF THE PREFERRED EMBODIMENTS OF THE INVENTION

Applicants have discovered that certain mutant forms of HSV-TK, which have been described by others (Black et al., 1996; U.S. Pat. No. 5,877,010 to Loeb et al., which is incorporated herein by reference in entirety) can be used to effect significant improvements in the radiosensitization of cancer cells. The mutants were initially generated in an attempt to improve gene therapy cancer treatment protocols which utilize HSV-TK but which do not employ radiation therapy. In these mutants, the active site has been “remodeled” in an attempt to increase the substrate specificity of the enzyme towards the guanosine nucleoside analogs GCV and ACV and/or to decrease utilization of thymidine. (Wild-type HSV-TK preferentially phosphorylates thymidine which thus acts as a strong competitive inhibitor of the enzyme.) Several mutants were identified which, compared to HSV-TK, displayed enhanced ratios of activity for phosphorylation of ACV and/or GCV in vitro compared to phosphorylation of thymidine. Some mutants displayed higher levels of in vitro activity for ACV or GCV when compared to wild type but retained high levels of thymidine utilization; others displayed lower levels of in vitro ACV/GCV activity but much lower levels of thymidine utilization. Variability was also observed in the level of expression of the mutants in cultured mammalian cells. For example, the mutant HSV-TK75 was expressed at only half the level of wild type HSV-TK in cultured hamster cells (Black et al. 1996). The applicability of the mutants to treating tumors was suggested but not demonstrated by either Black et al. or Loeb et al. There was, however, no suggestion of adapting the mutants for use in the radiosensitization of cancer cells or tumors.

As discussed in the background section, it is known that cancer cells can be radiosensitized by transfection or transduction (the nucleic acid is introduced into cells with the help of a virus vector) of the HSV-TK gene followed by exposure to a prodrug. The exact mechanism of such radiosensitization is unknown. However, it is known that the prodrug is converted into a phosphorylated species (active drug) that then enters the cellular nucleoside pool which is used as building blocks for DNA synthesis and replication. Upon the incorporation of the active drug into the DNA, a single-strand break results, which is toxic to the cell. It is believed, but not proven, that such single-strand DNA breaks are targets for radiation which converts the single-strand break into a double-strand break that is lethal to the cell.

Because of the lack of understanding of the mechanism of HSV-TK radiosensitization, methods of optimization are not necessarily straightforward. However, Applicants have discovered that certain of the HSV-TK mutants described by Black et al. and Loeb et al.,(in particular, HSV-TK75, a mutant in which isoleucine and phenylalanine at positions 160 and 161 are both substituted with leucine, and amino acids alanine and leucine at positions 168 and 160 are substituted by valine and methionine, respectively), are capable of significantly improving the radiosensitization of cancer cells. This is in spite of the fact that, while the mutant displays enhanced activity for phosphorylating ACV, it retains a relatively high affinity for thymidine (Black et al., 1996). Further, as demonstrated by the data presented in the Examples section of the instant application (see below), this mutant displays only a marginally improved effect over wild-type HSV-TK (in combination with ACV) with respect to the in vivo treatment of tumors without the application of radiation. Thus, the toxicity effect is not merely additive, in that cancer cell killing by radiation therapy in conjunction with HSV-TK75/ACV is more effective than would be predicted from the levels of cell killing that can be achieved by the independent application of either therapy alone.

The present invention in its broadest sense provides novel, unobvious methods of radiosensitizing cancer cells and tumors, killing cancer cells and suppressing the growth of tumors, and treating cancer in a mammal. Each of these methods includes a step of contacting cancer cells or a tumor with a vector encoding a form of TK that displays increased nucleoside analog phosphorylation activity when compared to wild type HSV-TK. All three methods also comprise the step of administering a prodrug to the cancer cells or tumor. The methods of killing cancer cells and suppressing the growth of tumors and treating cancer in a mammal also include the step of delivering radiation to the cancer cells or tumor.

By “increased nucleoside analog phosphorylation activity when compared to wild type HSV-TK” we mean that, when measured under typical laboratory conditions, the enzymatic forms of TK which are utilized in the methods of the instant invention possess a level of activity for nucleoside analog phosphorylation that is higher than that of wild type HSV-TK. That is to say, when such a high activity form of TK is assayed under the same in vitro conditions as wild type HSV-TK (e.g. equal concentrations of enzyme, identical assay conditions, and the like) the high activity form of will convert more substrate (e.g. a nucleoside analog such as acyclovir or ganciclovir, or both) to phosphorylated product per unit time than does wild type HSV-TK. In a preferred embodiment of the present invention, the activity of the high activity form of TK is about at least 5% greater than that of HSV-TK. In yet another preferred embodiment, the activity of the high activity form of TK is about at least 20% greater than that of HSV-TK. Those of skill in the art are well-acquainted with comparative enzyme assays and will recognize that standard laboratory conditions involve such factors as the standardization of reaction conditions, concentrations of enzyme, appropriate use of controls, and the like. The increased level of activity may be due to any of several factors, or to a combination of several factors such as increased binding affinity for the nucleoside analog substrate, decreased affinity for inhibitors (such as nucleosides), greater thermal or proteolytic stability, increased solubility, ability to cross the BBB, more favorable kinetic parameters (e.g. specific activity, Km or kcat values) and the like.

With respect to in vivo activity against cancer cells and tumors, the high activity forms of TK that are utilized in the practice of the present invention may or may not display increased anti-tumor activity in vivo compared to wild type HSV-TK when utilized without radiation. For example, in Example 6 of the instant application, Applicants provide data demonstrating that the mutant HSV-TK75 does not display a significant increase in antitumor activity compared to wild type HSV-TK without radiation, as evidenced by the non-significant difference in survival time between rats treated with either AdCMV-TK or AdCMV-TK75.

In some preferred embodiments of the present invention, the high activity form of TK is a mutant of HSV-TK. By “mutant of HSV-TK” we mean a form of the enzyme that differs in primary amino acid sequence from the “wild type” form of the type 1 strain HSV-TK enzyme, the sequence of which is known and readily available (Gene Bank Accession # CAA32315). Mutants of a wild type form may be those in which a substitution of amino acids has occurred (either conservative or non-conservative), or a deletion or addition of one or more amino acids has occurred, and the like. Such changes in the primary structure of an enzyme may reflect mutations that have occurred in the genetic sequence that encodes the enzyme. Alternatively, such changes may be the result of mRNA processing, or of post-translational modifications including, for example, proteolytic or chemical cleavage. The term “mutant” also encompasses DNA or RNA genetic sequences that encode a “mutant” enzyme. Due to the redundancy of the genetic code, more than one genetic sequence can code for the same enzyme primary sequence. Such mutations in the genetic sequence that encodes an enzyme may be the result of natural processes and isolated from nature, or they may be the result of genetic manipulations via bioengineering or other laboratory techniques which are well-known to those of skill in the art, e.g. chemical- or radiation-induced mutations, and the like. The HSV-TK mutant enzymes that are utilized in the practice of the present invention may be the result of any process so long as they display increased nucleoside analog phosphorylation activity in comparison to wild type HSV-TK.

In a preferred embodiment of the present invention, the high activity form of TK is a mutant form of HSV-TK. However, those of skill in the art will recognize that many TK enzymes are known to those of skill in the art, and that any TK enzyme or mutant form of a TK enzyme which displays increased nucleoside analog phosphorylation activity compared to wild type HSV-TK may be used in the practice of the present invention. For example, other TKs from eukaryotic organisms (mammalian, yeast or fungi) or their viruses, such as other strains of herpes simplex virus, vaccinia, Epstein Barr, and Varicella Zooster virus; prokaryotic organisms including Escherichia coli, Salmonella and their phages, including the T-even phages, T2, T4, T6, and the like, may be used in the practice of the present invention. Those of skill in the art will recognize that various avenues exist for obtaining such forms of TK, including producing the enzyme from an organism that is available through commercial sources such as the American Type Culture Collection (Manassas, Va.) either for direct use or for mutagenesis, or obtaining the enzyme itself from a commercial source.

HSV-TK mutants with increased activity are also described in international patent WO9729196 to Couder et al. (published Aug. 14, 1997) which is hereby incorporated by reference in its entirety.

In preferred embodiments of the present invention, the high activity forms of TK that are utilized in the practice of the present invention are functional proteins which are expressed within or in proximity to targeted cancer cells. The functional proteins are expressed via transcription and/or translation of an appropriate nucleic acid sequence (e.g. DNA or RNA) which encodes the high activity TK. In the practice of the present invention, such a nucleic acid sequence may comprise a portion of a suitable vector which allows delivery of the nucleic acid sequence to a site within or in proximity to the targeted cancer cells, and which also allows the expression of a functional form of the high activity TK protein in or near the targeted cells. Expression may occur in or near the targeted cells because of the well-known “bystander effect” that is observed with prodrugs. While the activated form of the prodrug must be within a cell in order to exert its effect, it is believed that it is not necessary that the TK enzyme itself be expressed within the cell. Without being bound by theory, it is possible that expression of the high activity form of TK within or in proximity to some but not all of the targeted cancer cells or tumor cells will be sufficient to insure phosphorylation of the prodrug, which then can enter other “bystander” cells in the surrounding area. The exact location of expression of the high activity form of TK in this regard is not crucial to the practice of the methods of the present invention.

In preferred embodiments of the present invention, the vector is an adenoviral vector. In a preferred embodiment of the present invention, the adenoviral vector is AdCMV-TK75. However, those of skill in the art will recognize that many suitable vectors exist which can be utilized in the practice of the present invention. Examples of such vectors include but are not limited to plasmids, retroviral vectors derived from mouse or human (including human immunodeficiency virus), adenovirus-associated vectors, vectors derived from HSV, vectors derived from viruses associated with hepatitis, and modifications thereof Any vector which is capable of delivering a nucleic acid sequence encoding a high activity form of TK to an appropriate location (e.g. within or in proximity to a targeted cancer cell or tumor) such that a functional form of the mutant protein is expressed at that location, may be utilized in the practice of the present invention.

In the practice of the present invention, cancer cells or tumors are contacted with a vector encoding a high activity form of TK. The quantity of vector that must be delivered to the cancer cells will vary from one situation to another. For example, when used in the treatment of cancer in a mammal, the quantity of vector to be utilized will vary depending on various factors such as the type and location of cancer being treated, the physical condition of the mammal (including size, weight, overall state of health, gender, and the like) the level of activity of the high activity form of TK, the nature of the vector, and the like. In general, however, the quantity or dose of vector to be delivered will be in the range of about 10⁶ to 10¹³ virus particles, and preferably about 10¹¹ particles. Those of skill in the art will recognize that the details of optimizing the administration of gene therapy vectors to mammals, for example humans, are typically worked out during extensive animal testing, followed by clinical trials. The procedures to be followed for optimization are well-established and familiar to those of skill in the art. See, for example, Trask et al. 2000 and Kuri et al., 2000.

In addition, those of skill in the art will recognize that many additional means exist which are also suitable for the delivery of nucleic acid sequences to cells, including but not limited to direct injection of the nucleic acid, intracranial delivery, intratumoral delivery, intravenous delivery, delivery via ocular drops or inhalation, and the like. Further, the compositional form of the nucleic acid which is administered may differ as is suitable for the type of delivery, for example, the nucleic acid may be complexed with a polycation or with a metal ion, combined with lipophilic substances, as a DNA-antibody complex for cell-specific delivery, and the like. Any suitable means of delivering a nucleic acid sequence to a cell and any suitable compositional form of the nucleic acid may be utilized in the practice of the present invention. Further, the nucleic acid may be either DNA or RNA, or a DNA-RNA chimera, and may be generated synthetically or by any of a variety of laboratory techniques which are well-known to those of skill in the art.

In a preferred embodiment of the present invention, the expression of the gene encoding the high activity form of TK is under control of the cytomegalovirus (CMV) promoter. However, those of skill in the art will recognize that many other promoters exist which could also be utilized to control expression of the gene by a vector. Examples include but are not limited to: other virus-derived promoters such as Rous Sarcoma Virus (RSV); SV40 promoter, adenovirus promoter (such as major late promoter, MLP), retrovirus promoters such as those from MoMLV and Friend MuLV; tissue-specific promoters such as vascular endothelial growth factor (VEGF); brain-specific promoters such as glial fibrillary acidic protein (GFAP); tumor-specific promoters, such as carcino-embryonic antigen promoter, DF3/MUC1 promoter, tyrosine hydroxylase promoter; or radiation-inducible promoters, such as those based on Egr-1, c-jun, c-fos, and other immediate-early promoters from human or other mammals, and the like.

The delivery of a vector or nucleic acid may be carried out by any of a variety of suitable means which are well-known to those of skill in the art, including but not limited to topical, oral, parenteral, intranasal, intravenous, intramuscular, subcutaneous, intraocular, transdermal, intratumoral (directly into tumor) or intracerebral (directly into the brain) delivery, or by continuous delivery from implants (cells or containers; osmotic pumps or Omaya reservoirs), or radiation device implants used during brachytherapy, and the like. Those of skill in the art will further recognize that the inclusion of other appropriate substances conducive to the delivery route may also be warranted. For example, lipids may be included for transdermal delivery; various pharmaceutically acceptable formulations may be utilized for injectable preparations; and various stabilizers or other suitable materials may be included together with the DNA or vector as warranted. In a preferred embodiment of the present invention, the vector is delivered intratumorally.

In a preferred embodiment of the present invention, the vector or nucleic acid is delivered to the targeted cancer cells in vivo. However, the vector or nucleic acid may also be inserted into host cells ex vivo, and the host cells may then be transplanted into a patient in need of treatment.

The practice of the present invention also involves administration of a prodrug. The prodrug may be administered in any of a variety of suitable means, including but not limited to topical, oral, parenteral, intranasal, intravenous, intramuscular, subcutaneous, intraocular, transdermal, intratumoral, intracerebral delivery, and the like. In a preferred embodiment, the prodrug is delivered orally or intravenously.

The quantity of prodrug to be delivered will vary from case to case, depending on various factors such as the gender, sex, age, and physical condition of the patient, the type of cancer being treated, the location of the cancer, and the like. In a preferred embodiment of the present invention, the quantity of prodrug to be delivered will, however, be in the range of 5-30 mg/kg body weight/day, and preferably 10 mg/kg trice daily. Those of skill in the art will recognize that the details of drug administration are normally worked out according to well-established protocols during clinical trials.

The sequence of delivery of a nucleic acid sequence encoding a TK mutant, administration of a prodrug, and provision of radiation therapy can also vary from patient to patient. However, in general, the nucleic acid sequence will first be delivered, for example, on day one; the prodrug will be administered, on, for example, day two or three and will continue for between 7-14 days; and the radiation treatment will be provided either simultaneously or following prodrug administration.

The present invention provides a method to radiosensitize cancer cells and tumors. For example, by practicing the methods of the present invention, the cancer cells which make up a tumor in a mammal may be effectively killed by lower (and less toxic to normal cells) or less frequent doses of radiation than are currently used in typical radiation therapy protocols. Or, the typical regimens of radiation therapy may be utilized with the methods of the present invention, and those regimens may result in more effective killing (e.g. fewer cancer cells survive the radiation) than would usually occur. The protocols for traditional radiation therapy, e.g. γ-radiation, are known, readily available, and routinely practiced by those of skill in the art. These include established protocols for the administration of drugs in combination with radiation therapy (Wobst et al., 1998. Ann. Oncol. 9, 951-962).

The type, dose, and frequency of ionizing radiation which is to be delivered in order to kill the targeted cancer cells according to the methods of the present invention or to treat cancer in a mammal will vary depending on a variety of factors including but not limited to: the intensity and source of the radiation, the location of the cancer cells, the type of cancer cells, relevant factors regarding the mammal that is being treated (e.g. gender, height, weight, age, general physical condition, stage of the disease, and the like), other elements of the treatment protocol (e.g. other drugs that are being administered, complementary surgical procedures, etc.), and the like. These factors will vary from case to case and are best determined by a skilled practitioner such as a physician. The usual practice with respect to the development of cancer therapies is that the details of the therapies are based on protocols which are well-known to those of skill in the art, and that the final details regarding the novel aspects of innovative protocols are defined during the well-established procedures of clinical trials. Typical radiation sources include but are not limited to: those clinically-used radioisotopes such as ⁶⁰Co, ¹³⁷Cs; devices such as x-ray tubes, gamma rays, linear accelerators, neutrons, protons, gamma-knife, brachytherapy (implantation of radioactive seeds); or systemically, intracerebrally or intratumorally delivered radionuclides (I¹²⁵, I¹³⁵, I¹⁹²) attached to natural molecules such as antibodies or lipids for cell or tumor targeting; or encapsulated in beads or containers; and the like. Typical radiation protocols include but are not limited to: conventional fractionated irradiation delivered by standard daily doses of 2 Gy or less; or by methods designed to spare radiation toxicity to normal tissues while maximizing tumor toxicity such as conformal radiation therapy, including three-dimensional planning and intensity-modulated radiation therapy (IMRT).

In preferred embodiments of the present invention, the radiation that is delivered to kill the targeted tumor cells is in the range of from about 1 to about 10 Gy. In a preferred embodiment of the present invention, the radiation dose is about 2 Gy or fraction thereof such that 2 Gy of radiation is delivered over the course of one day. Super-fractionated delivery protocols may prescribe greater than 2 Gy per day.

In a preferred embodiment of the present invention, the cancer cells which are radiosensitized and killed are glioma cells, gliomas and other types of malignant brain tumors. However, those of skill in the art will recognize that many other types of cancer cells and tumors can also be radiosensitized or killed by the methods of the present invention, and that many types of cancer can be treated by the methods of the present invention. Examples include but are not limited to: breast cancer, prostrate cancer, lung cancer, skin cancer (melanoma), head and neck cancer, and the like. Any type of cancer which can be ameliorated by the methods of the present invention may be treated by the methods of the present invention.

The methods of the present invention involve administration of a prodrug. In a preferred embodiment of the present invention, the prodrug is acyclovir (ACV). However, those of skill in the art will recognize that many other types of prodrugs exist which are suitable for use in the practice of the present invention. For example, ganciclovir (GCV), trifluorothymidine, 1-[2-deoxy, 2-fluoro, β-D-arabinofuranosyl]-5-iodouracil, Ara-A, Ara-T, 1-β-D-arabinofuranosyl thymidine, 5-ethyl-2′-deoxyuridine, iodouridine, AZT, AIU, dideoxycytidine, Ara C, bromodeoxycitidine, iododeoxycytidine, bromovinyldeoxyuridine, and the like. Any prodrug which is a substrate for a form of TK that is suitable for use in the present invention may be utilized in the practice of the present invention, so long as the combination of the TK and the prodrug are conducive to the radiosensitization of cancer cells. For example, if the TK exhibits increased activity toward the substrate acyclovir, then acyclovir may be administered. If the TK exhibits increased activity toward the substrate gangciclovir, then gangciclovir may be administered. Some forms of TK may exhibit enhanced activity toward more than one substrate. In those cases, any appropriate substrate, or a mixture of appropriate substrates, may be administered. Any combination of mutant TK and prodrug substrate may be administered so long as the result is the radiosensitization of cancer cells or tumors.

The present invention provides a method of treating cancer in a mammal. The method may be utilized alone or in concert with other types of cancer or related health treatments, for example, with chemotherapy, surgical removal of tumors, dietary supplements, substances administered for the control of pain, and the like.

In the Examples below, we demonstrate the following:

1) Example 1: AdCMV-TK and AdCMV-TK75 express the 44-kDa HSV-TK protein at similar levels in rat RT2 glioma cells;

2) Example 2: in vitro, mutant HSV-TK75 is more effective than wild type HSV-TK in killing rat RT2 glioma cells in combination with either GCV or ACV;

3) Example 3: in vitro, mutant HSV-TK75 in combination with ACV performs significantly better than wt HSV-TK in radiosensitizing rat RT2 cells at clinically relevant radiation doses;

4) Example 4: in vitro, ACV treatment in combination with radiation significantly increases apoptosis in cells infected with HSV-TK adenovirus;

5) Example 5: in vivo, treatment with a combination of wild type AdCMV-TK transduction and exposure to ACV radiosensitizes rat glioma and almost doubles the survival time of AdCMV-TK-treated animals compared to control rats;

6) Example 6: in vivo, treatment with a combination of mutant type AdCMV-TK75 transduction and exposure to ACV does NOT significantly prolong the survival time of mutant AdCMV-TK75 treated animals compared to animals treated with wild type AdCMV-TK;

7) Example 7: in vivo, treatment with a combination of mutant type AdCMV-TK-75 transduction and exposure to ACV results in significantly superior radiosensitization of rat glioma compared to wild type HSV-TK. Most importantly, the presence of HSV-TK75 alone did not ensure significantly longer survival. Only the combination of the presence of HSV-TK75 plus irradiation significantly prolonged survival.

Examples 6 and 7 serve to illustrate the unobvious and novel finding that, in vivo, treatment of tumors with high activity HSV-TK75 plus the prodrug ACV is successful only in combination with radiation. Treatment of tumors with HSV-TK75 plus ACV without radiation is no more successful than treatment with wild type HSV-TK plus ACV.

The following examples are included in order to provide a more complete understanding of various embodiments of the instant invention but should not be construed so as limit the practice of the present invention in any way.

### EXAMPLES

### Methods

Cell Culture

RT2 cells (a rat glioma cell line derived from Fischer 344 rats (Wilfong et al. 1973) that form tumors with features typical of human glioblastoma (Mahaley et al, 1977) were cultured in Dulbecco's modified Eagle's medium with 10% fetal bovine serum supplemented with penicillin/strepto-mycin. (Brust et al., 2000). Infected cells were exposed to Zovirax/ACV (manufactured by Glaxo Wellcome, Chapel Hill, N.C.) or GCV (kindly provided by Syntex, Palo Alto, Calif.) added to the medium. Cells were irradiated in medium with a ⁶⁰Co γ irradiator (Picker Zonegard V4 M60; Picker Interational, Cleveland, Ohio) set at 1 Gy/minute. Cell viability was determined by the 3-(4,5-dimethylthiazol-2-yl)-2,5-dimethyltetrazolium bromide (MTT) assay. (Contessa et al, 1999). Terminal deoxynucleotidyltransferase-mediated deoxyuridine triphosphate nick end labeling assays were carried out with an ApopTag Direct kit from Oncor (Gaithersburg, Md.), according to the manufacturer's instructions, to provide a measurement of apoptosis.

Adenovirus

Adenovirus was made in bacteria essentially as described elsewhere.(Chartier et al, 1996). Mutant HSV-TK75 cDNA and the corresponding wt gene were kindly provided by Margaret Black (University of Washington, Seattle, Wash.).(Black et al., 1996). To make adenovirus that expresses wt or mutant HSV-TK75 under control of the cytomegalovirus promoter, we cloned a 1.4-kb NcoI fragment from either pET23d:HSVTK or pET23d:75 7 into the BamHI site of pZERO-TGCMV by blunt-end ligation after filling in the 5′-overhangs with Klenow polymerase. In the process, both the NcoI and BamHI sites are restored. Adenovirus (dLE1, dLE3) was then made in Escherichia coli as described previously (Chartier et al, 1996). Adenovirus expressing wild type HSV-TK (AdCMV-TK), mutant HSV-TK75 (Ad-CMV-TK75), and AdCMV (empty vector) were grown in 293 cells permissive for growth of El-deleted Ad-5 (Graham et al., 1995) as described previously (Valerie and Singhal, 1995) and purified by double CsC1 gradient centrifugation followed by dialysis against 13% glycerol in phosphate-buffered saline as described (Valerie and Singhal, 1995; Valerie, 1999). Virus was snap frozen in liquid nitrogen and stored at −70° C. until further use. Virus titering was done using a direct immunofluorescence assay (Light Diagnostics, Temecula, Calif.) and a standard plaque assay. Titers of 2×10¹¹ plaque-forming units/mL were routinely achieved.

Protein analysis

Western blot analysis was carried out essentially as described previously (Taher et al, 1999) with rabbit polyclonal anti-HSV-TK antibody (Ab) at a 1/5000 dilution followed by a 1/2000 dilution of goat anti-rabbit immunoglobulin G coupled to alkaline phosphatase (Oncogene Science, Manhasset, N.Y.) and developing with CDP-Star (Tropix, Bedford, Mass.).

Animals

Female Fischer 344 rats (150-160 g) were purchased from Harlan Sprague Dawley. Animals were anesthetized and immobilized with a single intra-peritoneal injection of a Ketamine-HCl (50 mg/ml):Xylazine (10 mg/ml) solution.

Intracerebral Implantation of RT2 Cells

Transduced RT2 cells were implanted intracerebrally using a stereotaxic frame (Stoelting, Wood Dale, Ill.). Briefly, 3×10⁴ cells in 5 ml infected 48 h earlier with multiplicity of infections (MOIs) ranging between 3 and 30 were implanted in the right parietal lobe, 3 mm to the right and 1 mm posterior to the bregma. Transduced cells were delivered with a 28-gauge infusion needle lowered to a depth of 3.5 mm to place the tip of the needle in the caudate putamen to avoid the ventricular space. This location avoids back flow of the infusate preventing escape along the needle track. Upon completion of injecting the tumor cells, the needle was withdrawn, the burr-hole sealed with bone wax and the incision closed with a wound clip. Osmotic mini-pumps (ALZET model 2ML1, ALZA Scientific Products, Palo Alto, Calif., delivering 10 ml/hr over 7 days) were filled with 2 ml of a 50 mg/ml solution of Zovirax®/acyclovir (ACV) from Glaxo-Wellcome, Chapel Hill, N.C. The osmotic pumps were implanted sub-cutaneously between the scapula immediately following implantation of cells. Animals were returned to their cages and allowed to eat and drink ad libitum. Irradiations started on day 5 after implantation. Animals were anesthetized as described above for surgery, put in a shielding block and the head exposed to 7.25 Gy delivered from a Picker Zonegard V4M60 ⁶⁰Co gamma irradiator at 1 Gy/min. Radiation exposures were repeated daily for 3 consecutive days (day 5-7 after implantation). Animals were monitored daily for signs of abnormal behavior, i.e., circling behavior indicative of bacterial infections, early and persistent lethargy and recumbent behavior indicative of cerebral hemorrhage, at which time animals were sacrificed.

Statistical Analysis

Statistical analysis was carried out by 3-way variance F-test analysis using the Splus (version 4.5) computer software. The statistical procedure of “analysis of variance” (Eiswehnart, 1947) was used to detect differences between the data sets. For the pair-wise comparison between means, t-test was used with adjusting the critical region of the test to accommodate the Bonnferoni correction (Miller, 1981).

### Example 1

### Construction and Testing of an Adenovirus Expressing Mutant HSV-TK75

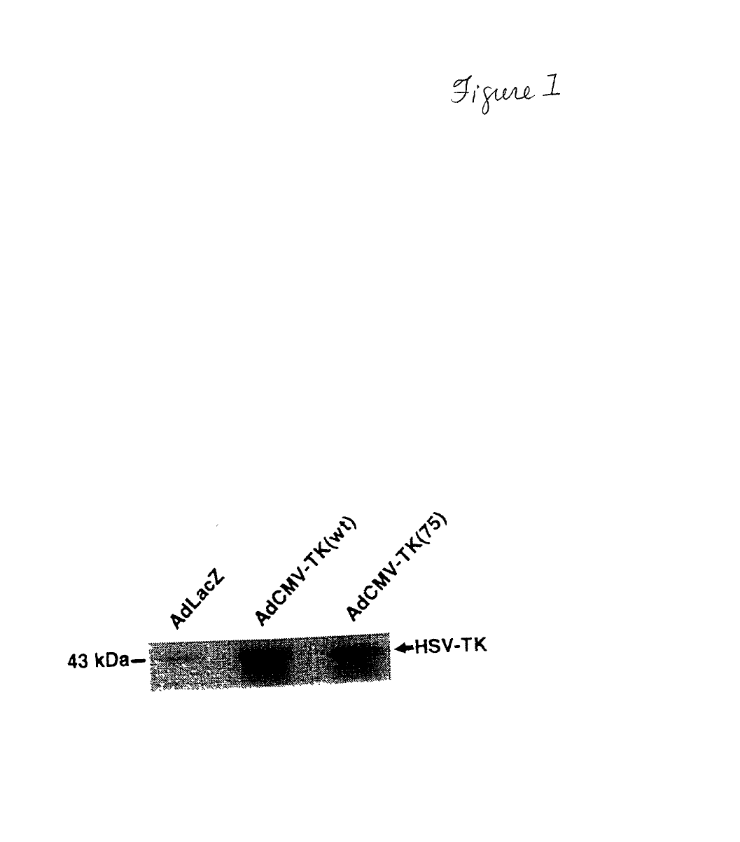

In stably transfected hamster cells, mutant HSV-TK75 protein levels are expressed at only approximately half those of wild type HSV-TK.(Black et al, 1996). To test for the expression of HSV-TK in mammalian cancer cells, we infected rat RT2 glioma cells with an adenovirus expressing the β-galatosidase reporter (Brust et al., 2000), or the newly constructed AdCMV-TK and AdCMV-TK75 virus. This experiment was necessary to confirm expression of the mutant HSV-TK in mammalian cancer cells, and to determine whether previously observed differences in toxicity between wild type and mutant HSV-TK75 viruses (Black et al, 1996) might be attributable merely to the differential expression/steady-state level of HSV-TK between the two viruses. Using a polyclonal rabbit Ab raised against HSV-TK in a Western blot experiment, we were able to demonstrate that AdCMV-TK and AdCMV-TK75 expressed the 44-kDa HSV-TK protein at similar levels in RT2 glioma cells (FIG. 1).

This Example demonstrates that AdCMV-TK and AdCMV-TK75 express the 44-kDa HSV-TK protein at similar levels in RT2 glioma cells in vitro.

### Example 2

### Mutant HSV-TK75 Predisposes RT2 Cells to Killing by GCV and ACV More Efficiently Than Wild Type HSV-TK in vitro

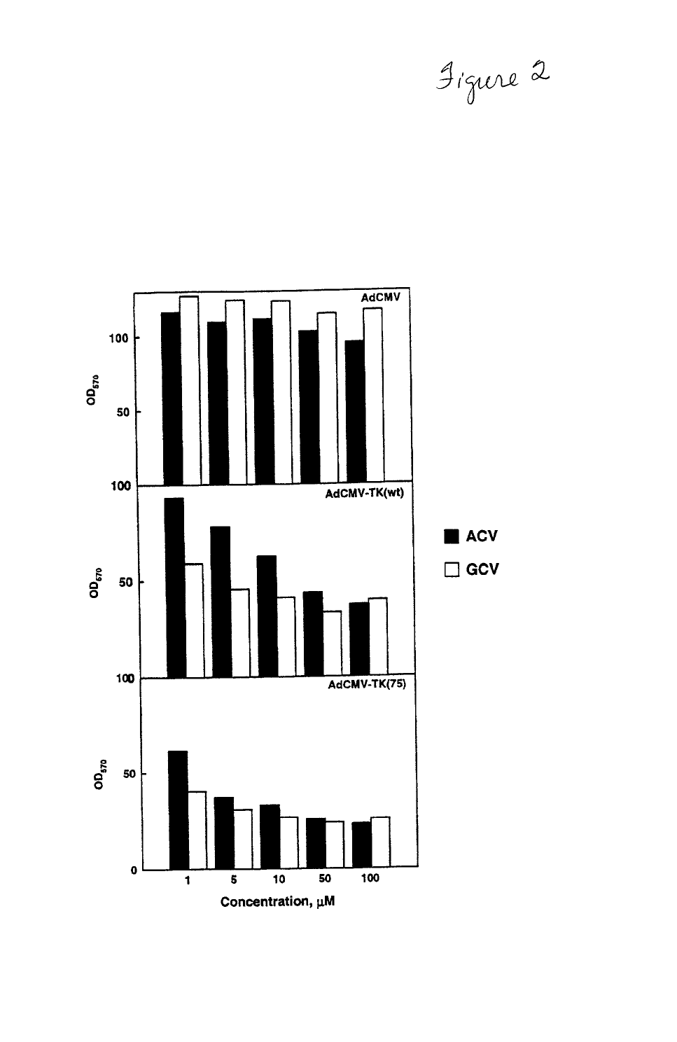

It was subsequently determined whether infection of RT2 cells with the adenovirus expressing mutant HSV-TK 75 was able to predispose RT2 cells to killing by GCV and ACV more efficiently than cells infected with virus expressing wild type HSV-TK. At 2 days postinfection, infected cells were exposed to increasing concentrations (1-100 microM) of either GCV or ACV. As a control, we used an adenovirus(AdCMV) carrying no transgene. We found that GCV or ACV had little to no effect on control AdCMV-infected RT2 cells (FIG. 2, top). Infection with AdCMV-TK resulted in significant cell killing with both GCV and ACV (FIG. 2, middle). Cell killing with GCV was more effective than with ACV, in agreement with a lower K m for GCV (Balzarini et al, 1993; Field et al, 1983). Finally, we found that mutant HSV-TK 75 predisposed RT2 cells to killing by GCV and ACV better than HSV-TK (FIG. 2, bottom).

These results demonstrate that mutant HSV-TK75 is more effective than wild type HSV-TK in killing RT2 glioma cells in vitro in combination with either GCV or ACV.

### Example 3

### Mutant HSV-TK75 in Combination with ACV Radiosensitizes RT2 Cells More Efficiently Than Wild Type HSV-TK in vitro

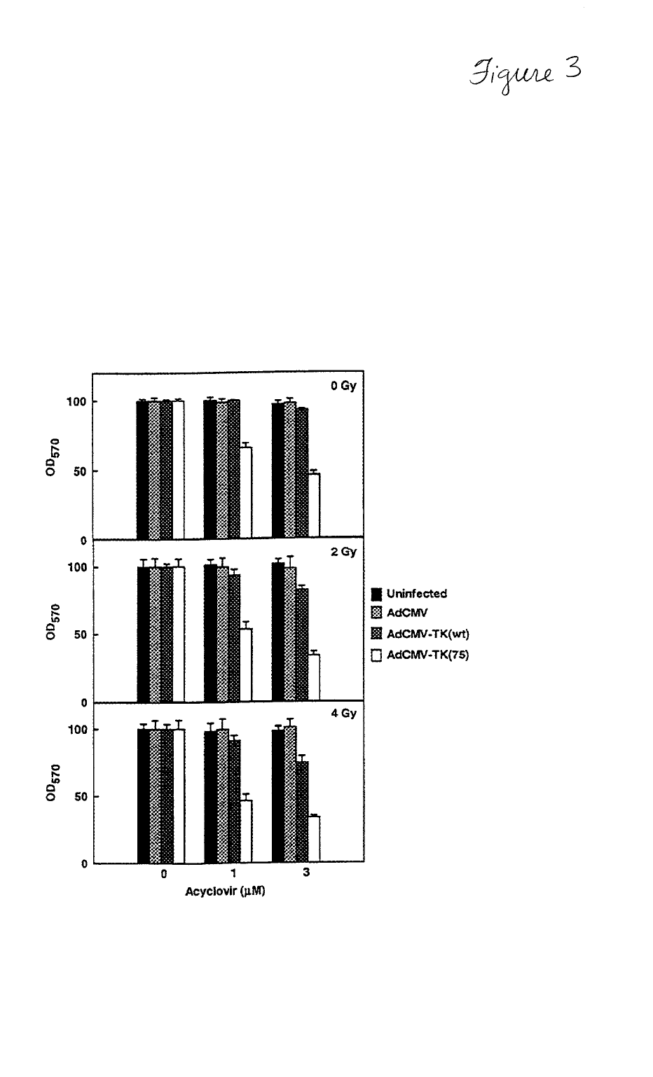

To determine whether mutant HSV-TK75 was also more effective than wild type virus as a radiosensitizer in combination with ACV, we infected RT2 cells and after 2 days, provided ACV at either 1 or 3 microM. After another day, the cells were exposed to either 2 or 4 Gy of radiation followed by cell proliferation/survival assay 5 days later. Without any radiation, it was observed that Ad-CMV-TK did not predispose the cells to killing by ACV significantly, whereas HSV-TK75 reduced cell growth by ˜25% at 1 microM and 50% at 3 microM (FIG. 3, top). Radiation caused further killing of both wt HSV-TK- and HSV-TK75-infected cells in a dose-dependent fashion (FIG. 3, middle: 2 Gy; FIG. 3, bottom: 4 Gy). The sensitizer enhancement ratios (SERs) were calculated (Table 1). A significant (P<0.0001) difference between the SER obtained with wild type and mutant HSV-TK75 is notable, in particular with the clinically relevant lower doses of 2 Gy and 1 microM ACV (1.1 vs. 1.4).

This result demonstrates that, in vitro, mutant HSV-TK75 expressed from an adenovirus performs significantly better than wild type HSV-TK in radiosensitizing RT2 cells in combination with ACV at clinically relevant radiation doses (2 Gy) and low (micromolar) concentrations of ACV which mimic those attainable in sera.

TABLE 1
Sensitizing enhancement ratios (SERs) of RT2 cells infected with
either AdCMV-TK or AdCMV-TK75

| ACV | 2Gy | | 4 Gy | |
|---|---|---|---|---|
| (μM) | AdCMV-TK | AdCMV-TK75 | AdCMV-TK | AdCMV-TK75 |
| 1 | 1.1 | 1.4 | 1.2 | 1.6 |
| 3 | 1.2 | 1.5 | 1.4 | 1.5 |

Average values obtained from the data shown in FIG. 3 were used to determine SER values. SER is defined as the ratio between growth/survival without radiation divided by survival with radiation. Significant (P≦−0.0001) differences were found for the ACV and radiation dose effects for each of the two viruses, and, most importantly, between the AdCMV-TK and AdCMV-TK75 adenovirus.

### Example 4

### Mutant HSV-TK75 Potentiates Radiosensitization Through Increased Rate of Apoptosis

To establish the mode of death that occurs after RT2 cells are exposed to these treatments, we determined the level of apoptosis. As described above, RT2 cells were infected with either one of the two HSV-TK adenoviruses or an empty control virus (AdCMV) and then exposed to 3 microM ACV for 20 hours followed by low doses of radiation (2 or 5 Gy). The extent of apoptosis was determined 20 hours after radiation (40 hours after ACV treatment). We found that control-infected cells did not undergo significant apoptosis after exposure to ACV irrespective of radiation. Cells infected with AdCMV-TK underwent apoptosis after exposure to both ACV and radiation (4-6%), but not in the absence of radiation. However, cells infected with AdCMV-TK75 and exposed to ACV showed ˜9% apoptotic cells without radiation and ˜14% with either 2 or 5 Gy of radiation. No apoptosis was detected without ACV or radiation with any of the viruses.

This result demonstrates that ACV treatment in combination with radiation significantly increases apoptosis in cells infected with HSV-TK adenovirus. Note that an apoptotic mechanism of cell death also has been suggested for HSV-TK-mediated killing of glioma with the drug GCV (Colombo et al., 1995; Hamel et al, 1996). In addition, mutant HSV-TK75 performed significantly better, almost doubling the number of cells undergoing apoptosis compared with infection with wt HSV-TK virus. Most importantly, an ˜2-fold potentiation of drug-induced apoptosis with radiation was clearly noticeable with mutant HSV-TK75. Furthermore, little to no increase in apoptotic cells was noticed by increasing radiation from 2 to 5 Gy, suggesting that the potentiation effect has already peaked at 2 Gy. This finding is important because 2 Gy is the dose used clinically.

### Example 5

### In vivo Radiosensitization of RT2 Glioma with AdCMV-TK and ACV

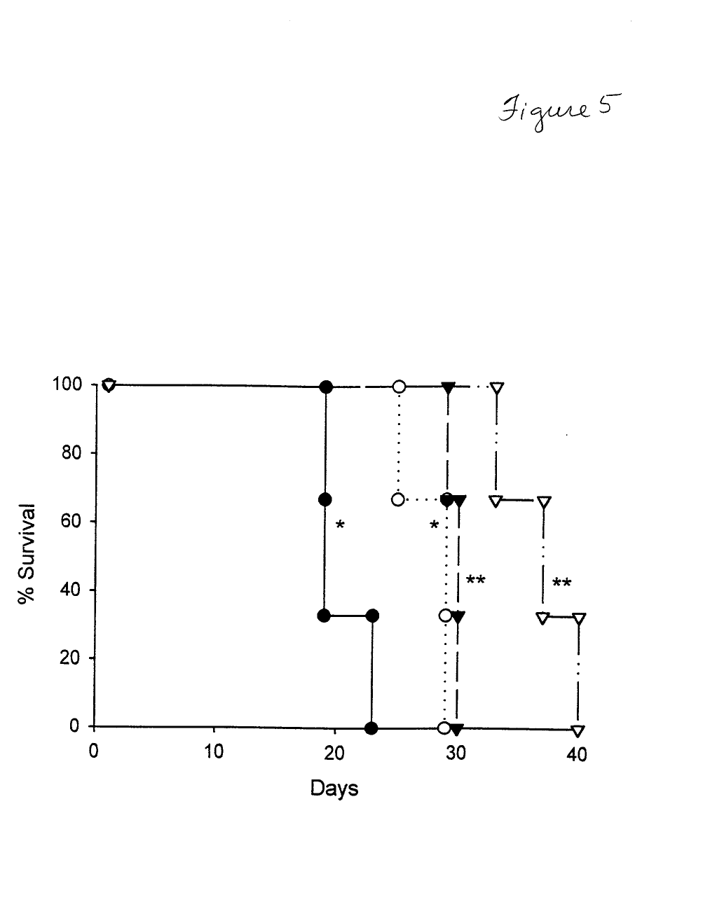

To examine whether adenovirus-expressed HSV-TK in combination with systemically administered ACV would sensitize glioma to radiation in vivo, 3×10⁴ cells transduced in vitro with either Adβgal or AdCMV-TK were cerebrally implanted into syngeneic Fischer 344 rats. An MOI between 10 and 30 transduced close to 100% of the RT2 cells (data not shown). Six animals were implanted with RT2 cells transduced with either one of the two viruses. Then, five days after implantation and ACV exposure (osmotic pump), the brains of three animals from each set were irradiated with 7.25 Gy on three consecutive days (FIG. 5). This irradiation protocol added approximately 10 days to the 20 days survival time of rats implanted with 3 ×10⁴ non-transduced RT2 cells (data not shown).

Significant differences in the mean survival times between the four groups (p<0.001) were found. The mean survival time of Adβgal-implanted rats exposed to ACV was ˜20 days. Fractionated irradiation (3×7.25 Gy) increased the survival of these animals to ˜28 days (p<0.025). Rats implanted with AdCMV-TK-transduced RT2 cells that were given ACV survived slightly longer (˜30 days) than rats implanted with Adβgal-transduced cells that were also irradiated. Finally, animals that received AdCMV-TK-transduced cells and were then irradiated, survived ˜37 days, significantly (p<0.02) longer than animals that were not irradiated.

This result clearly demonstrates that treatment with a combination of AdCMV-TK transduction and exposure to ACV radiosensitizes syngeneic rat glioma and almost doubles the survival time of AdCMV-TK-treated animals compared to Adβgal-treated control rats.

### Example 6

### Only Marginally Improved Therapeutic Effect with Mutant HSV-TK75 Over Wild Type HSV-TK in Combination with ACV

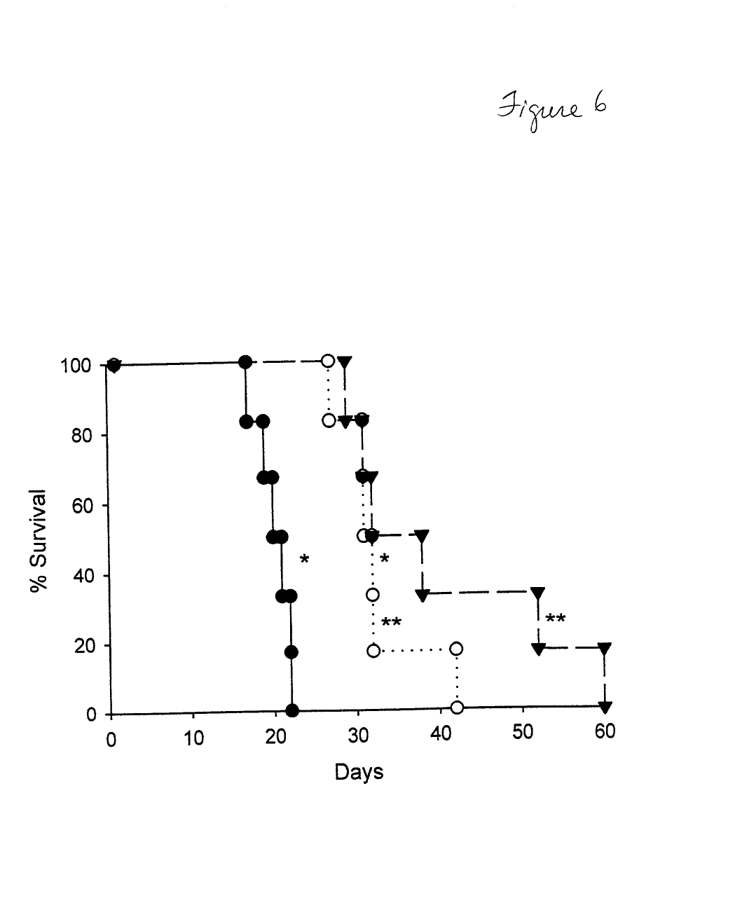

To examine whether enhanced killing was possible to achieve in vivo with the HSV-TK75 mutant, RT2 cells transduced in vitro with either Adβgal, AdCMV-TK, or AdCMV-TK75 virus 2 days earlier were again intracerebrally implanted. All animals received ACV from osmotic pumps, but were not given any radiation. The result of an experiment using an MOI of 30 is shown in FIG. 6. We found that there were significant differences in the mean survival between the three groups (p=0.002). In pair-wise comparisons, animals implanted with cells transduced with AdCMV-TK lived longer (˜33 days) than animals implanted with cells transduced with Adβgal (˜20 days), which was highly significant (p<0.001). However, rats implanted with cells transduced with the AdCMV-TK75 virus expressing mutant HSV-TK75, increased the survival to ˜40 days over rats implanted with AdCMV-TK-transduced cells, but this difference was not significant (p=0.19), because of tailing of the curve. Similar, non-significant trends were also observed when rats were implanted with cells with the lower MOIs of either 3 or 10 (data not shown), suggesting that this marginal effect was not related to the expression level of mutant HSV-TK75 protein or the MOI.

This result demonstrates that, in vivo, the survival of animals with implanted tumor cells is not significantly prolonged when the cells have been transduced with mutant HSV-TK75 compared to wild type HSV-TK adenovirus.

### Example 7

### Substantially Improved in vivo Radiosensitization with Mutant HSV-TK75 Over Wild Type HSV-TK in Combination with ACV

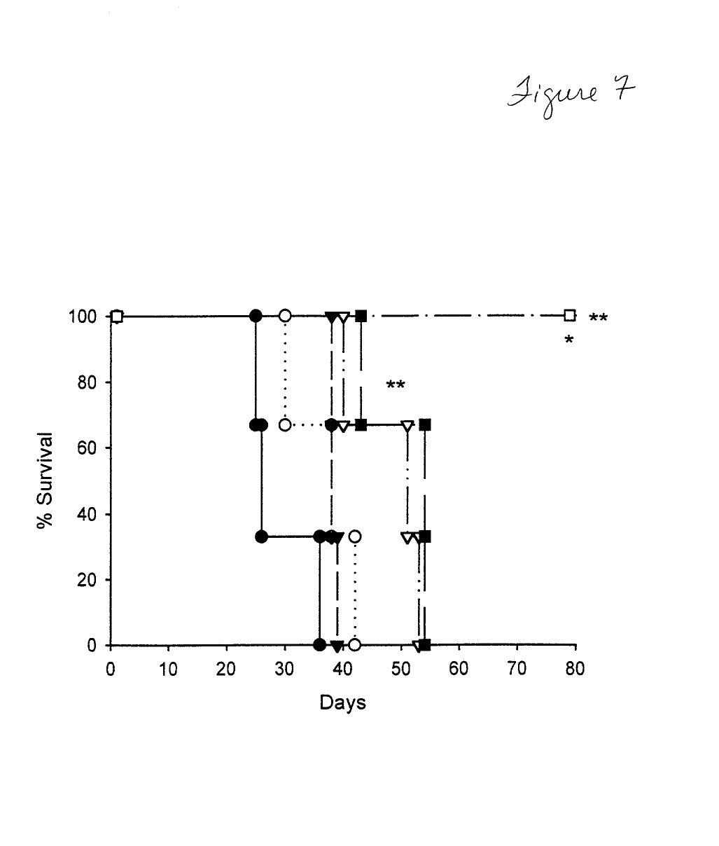

Next, to examine whether mutant HSV-TK75 improved radiosensitization of glioma compared to HSV-TK, transduced RT2 cells were implanted as described with either Adβgal, AdCMV-TK, or AdCMV-TK75 virus at an MOI of 30. All animals received ACV from osmotic pumps and half the number of animals from each group were irradiated (as in Example 5) on three consecutive days. The mean survival times of animals that were not irradiated were approximately 29, 38, and 50 days for Adβgal-, AdCMV-TK-, and AdCMV-TK75-treated animals, respectively (FIG. 7). Fractionated radiation increased the mean survival times for the three groups to 37, 48, and 80 days, respectively. The animals implanted with cells transduced with AdCMV-TK75 and then irradiated showed no signs of abnormal behavior and were sacrificed on day 80. We found significant (p<0.001) differences in the survival time of animals between the six groups. In pair-wise comparison, there was a significant (p=0.005) difference in survival between irradiated and unirradiated animals implanted with AdCMV-TK75-transduced cells. However, there were no significant pair-wise differences between any other two groups in this experiment, although the trends are the same as demonstrated in the two previous experiments., i.e. the presence of HSV-TK75 and prodrug alone did not ensure significantly longer survival. Only the combination of the presence of HSV-TK75 and prodrug plus irradiation significantly prolonged survival.

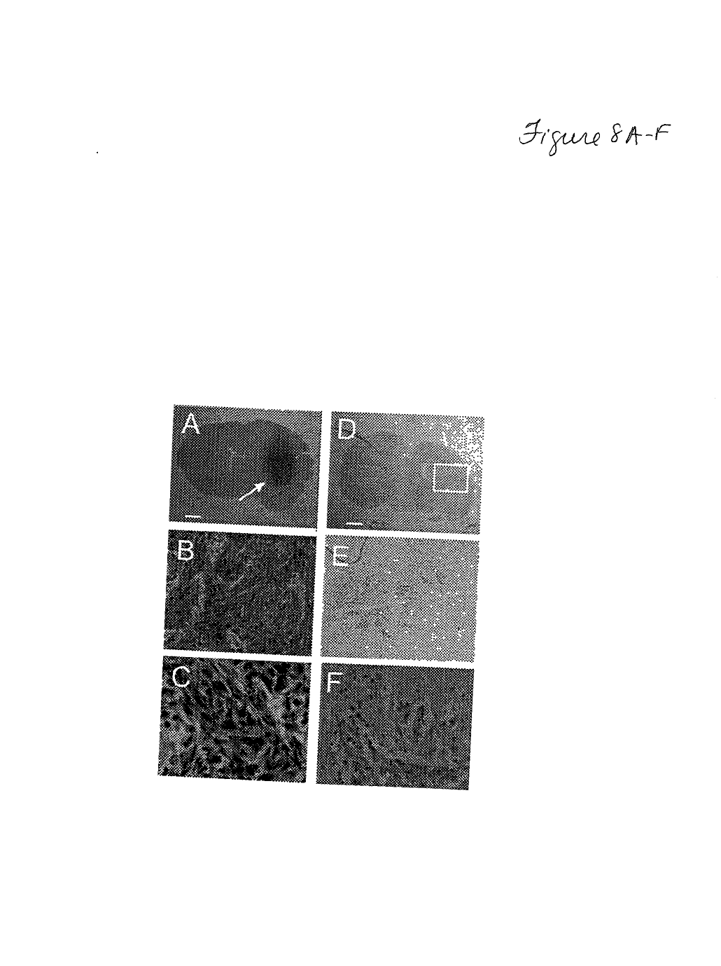

To establish the presence of tumor cells in an animal that we suspected might have succumbed to the growth of the implanted tumor cells, we sectioned the paraffin-embedded brain from an animal that had received Adβgal-transduced cell and stained the sections with hematoxylin-eosin (FIGS. 8A-F). As shown, at low power a tumor is easily distinguished as a darker stained area (FIG. 8A), and at higher power darker-stained nuclei of tumor cells and other infiltrating cell bodies can easily be distinguished (FIGS. 8B and 8C), suggesting that the cause of death of the Adβgal-treated rat was a brain tumor. We were also able to identify, however, with much more difficulty, a very small lesion in the brain of a sacrificed rat implanted with cells transduced with AdCMV-TK75 that was also given ACV and fractionated irradiation (FIGS. 8B, 8C, 8D). The presence of tumor cells was also evident in this animal but much less extensively. Consequently, the most likely reason for the sustained survival of animals treated with AdCMV-TK75 virus, ACV, and fractionated irradiation, was tumor growth inhibition.

### Example 8

### Colony-forming Ability of Human U87 Glioma Cells Transduced with AdCMV-TK, AdCMV-TK75, or Adβgal and Exposure to ACV Followed by Radiation

U87 cells were transduced with the appropriate virus at MOI=10, and two days later, trypsinized, diluted, and plated in 10-cm dishes. The cells were allowed to attach for several hours, then treated with 3 microM ACV. Twenty-four hours after addition of ACV, the dishes were irradiated (⁶⁰Co), and 24 h later the ACV removed, and the medium replaced with fresh media. The cells were then incubated for a further 12 days (14 days total since plating), stained with crystal violet, and colonies counted. In parallel, transduced cells were analyzed by Western blotting using anti-β-actin antibody (using chemiluminescence) and subsequently probed for HSV-TK (using CDP-Star), and shown to produce equal levels of BSV-TK (data not shown).

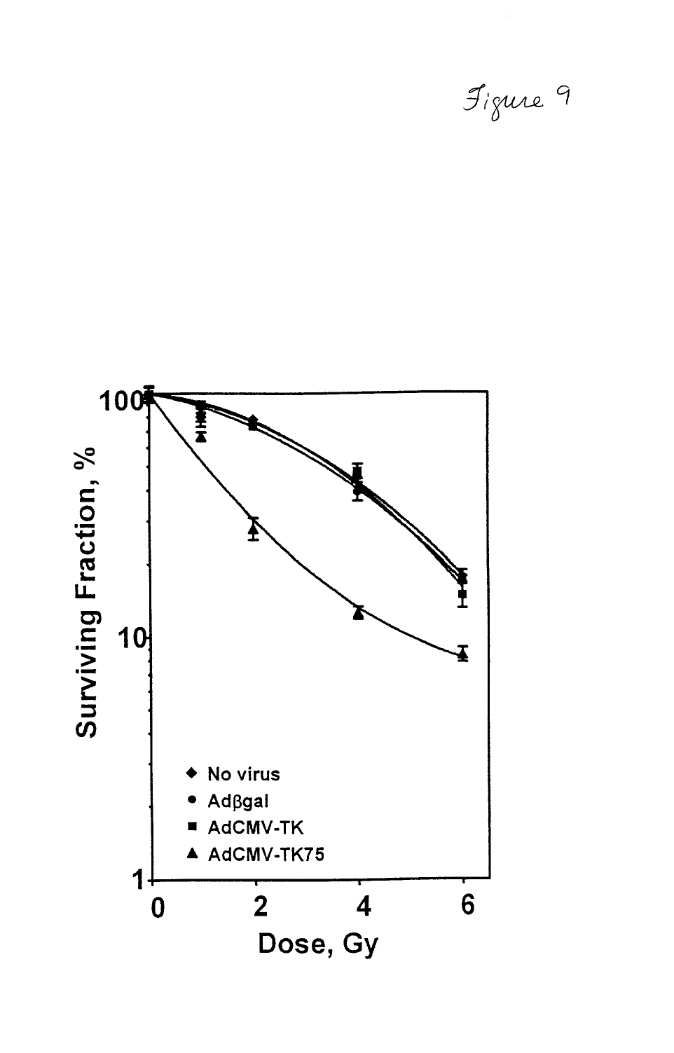

The results are given in FIG. 9 and demonstrate that human glioma U87 cells are radiosensitized after infection with AdCMV-TK75 expressing mutant HSV-TK75 at low concentrations of ACV (3 micromolar) which does not occur with cells infected with AdCMV-TK expressing wild type HSV-TK. Similar levels of HSV-TK and HSV-TK75 were produced by both cell populations, suggesting that it is the higher activity of HSV-TK75 toward lower concentrations of ACV and not protein levels that causes the increased radio sensitization. This result is in agreement with results generated with rat glioma cells (Examples 3-7) and attests that the combination treatment of AdCMV-TK-75 with low and clinically achievable concentrations of ACV is superior over treatment with AdCMV-TK and the same concentration of ACV.

### Example 9

### Radiosensitization of U87MG Xenografts Grown in the Flank of nu/nu Mice Transduced with AdCMV-TK75 or Adβgal and Administration of ACV

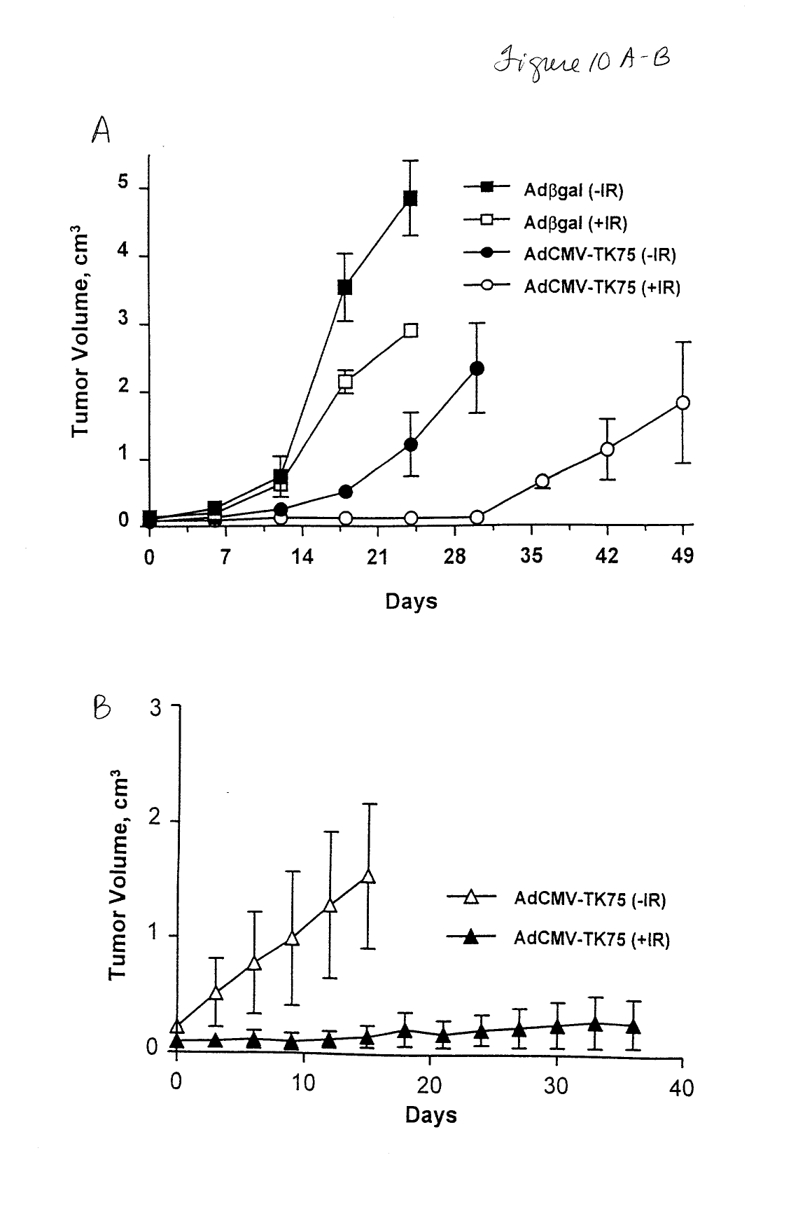

U87MG xenografts grown in nu/nu mice were infused with 2.2×10⁹ pfu/tumor of AdCMV-TK75 or Adβgal and treated with acyclovir (100 mg/kg BID) for 5 days and then treated with radiation on 3 consecutive days (3×10 Gy) following ACV administration (FIG. 10A). U87MG xenografts were infused with AdCMV-TK75 as above and treated with ACV (100 mg/kg BID for 5 days) concurrently with radiation (3×10 Gy) (FIG. 10B).

These results demonstrate that, in agreement with the results from the rat RT2/Fisher 344 glioma model (Example 8) human xenografts grown in nude mice are also radiosensitized by the administration of AdCMV-TK75 and ACV, suggesting that AdCMV-TK75 and ACV may also be a superior combination over AdCMV-TK and ACV to radiosensitize human glioma.

Altogether, these results unequivocally demonstrate that mutant HSV-TK75 is superior to wild type HSV-TK as a radiosensitizer of rat and human glioma cells in vitro and gliomas in vivo when administered together with ACV. This is in spite of the fact that only marginal, non-significant differences in survival rates were observed as a result of treatment with HSV-TK75 plus ACV compared to wild type HSV-TK plus ACV without radiation, as recounted in Example 6.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

### REFERENCES

Balzarini J, Bohman C, De Clercq E. Differential mechanism of cytostatic effect of (E)-5-(2-bromovinyl)-29-de-oxyuridine, 9-(1,3-dihydroxy-2-propoxymethyl)guanine, and other antiherpetic drugs on tumor cells transfected by the thymidine kinase gene of herpes simplex virus type 1 or type 2. J Biol Chem. 1993;268:6332-6337.

Black M E, Newcomb T G, Wilson H M, Loeb L A. Creation of drug-specific herpes simplex virus type 1 thymidine kinase mutants for gene therapy. Proc Natl Acad Sci USA. 1996;93:3525-3529.

Brust D, Feden J, Farnsworth J, et al. Radiosensitization of rat glioma with bromodeoxycytidine and adenovirus expressing herpes simplex virus-thymidine kinase delivered by slow-rate controlled positive pressure infusion. Cancer Gene Ther. 2000:7:778 -788.

Chartier C, Degryse E, Gantzer M, Dieterle A, Pavirani A, Mehtali M. Efficient generation of recombinant adenovi-rus vectors by homologous recombination in Escherichia coli. J Virol. 1996;70:4805-4810.

Colombo B M, Benedetti S, Ottolenghi S, et al. The “bystander effect”: association of U-87 cell death with ganciclovir-mediated apoptosis of nearby cells and lack of effect in athymic mice. Hum Gene Ther. 1995;6:763-772.

Contessa J N, Reardon D B, Todd D, et al. The inducible expression of dominant-negative epidermal growth factor receptor-CD533 results in radiosensitization of human mammary carcinoma cells. Clin Cancer Res. 1999;5:405-411.

Eiswehnart C. The assumptions underlying the analysis of variance. Biometrika 1947; 3:1-21.

Field A K, Davies M E, DeWitt C, et al. 9-([2-hydroxy-1-(hydroxymethyl)ethoxy]methyl) guanine: a selective inhibitor of herpes group virus replication. Proc Natl Acad Sci USA. 1983;80:4139-4143.

Graham F L, Smiley J, Russell W C, Nairn R. Characteristics of a human cell line transformed by DNA from human adenovirus type 5. J Gen Virol 1977; 36:59-74.

Hamel W, Magnelli L, Chiarugi VP, Israel M A. Herpes simplex virus thymidine kinase/ganciclovir-mediated apoptotic death of bystander cells. Cancer Res. 1996; 56:2697-2702.

Kim S H, Kim J H, Kolozsvary A, Brown S L, Freytag S O. Preferential radiosensitization of 9 L glioma cells trans-duced with HSV-tk gene by acyclovir. J Neurooncol. 1997; 33:189 -194.

Kim J H, Kim S H, Brown S L, Freytag S O. Selective enhancement by an antiviral agent of the radiation-induced cell killing of human glioma cells transduced withHSV-tk gene. Cancer Res. 1994;54:6053-6056.

Kim J H, Kim S H, Kolozsvary A, Brown S L, Kim O B, Freytag S O. Selective enhancement of radiation response of herpes simplex virus thymidine kinase-transduced 9 L gliosarcoma cells in vitro and in vivo by antiviral agents. Int J Radiat Oncol Biol Phys. 1995;33:861-868.

Khuri, F. R., Nemunaitis, J., Ganly, I., Arseneau, J., Tannock, I. F., Romel, L., Gore, M., Ironside, J., MacDougall, R. H., Heise, C., Randlev, B., Gillenwater, A. M., Bruso, P., Kaye, S. B., Hong, W. K., and Kim, D. H. a controlled trial of intratumoral Onyx-015, a selectively-replicating adenovirus, in combination with cisplatin and 5-fluorouracil in patients with recurrent head and neck cancer. Nat Med. 6: 879-85, 2000.

Mahaley M S Jr, Gentry R E, Bigner D D. Immunobiology of primary intracranial tumors. J Neurosurg. 1977;47:3543.

Miller R G, Jr. Simultaneous statistical inference. 2nd ed. New York: Springer-Verlag, 1981.

Taher M M, Baumgardner T, Sent P, Valerie P. Genetic evidence that stress-activated p38 MAP kinase is necessary but not sufficient for UV activation of HIV gene expression. Biochemistry. 1999;38:13055-13062.

Trask, T. W., Trask, R. P., Aguilar-Cordova, E., Shine, H. D., Wyde, P. R., Goodman, J. C., Hamilton, W. J., Rojas-Martinez, A., Chen, S. H., Woo, S. L., and Grossman, R. G. Phase I study of adenoviral delivery of the HSV-tk gene and ganciclovir administration in patients with current malignant brain tumors. Mol Ther. 1: 195-203, 2000.

Valerie K, Singhal A. Host-cell reactivation of reporter genes introduced into cells by adenovirus as a convenient way to measure cellular DNA repair. Mutat Res. 1995;336: 91-100.

Valerie K. Viral Vectors for Gene Therapy. In: Wu-Pong S, Rojanasakul Y, eds. Biopharmaceutical Drug Design and Development. Totowa, New Jersey: Humana Press, Inc., 1999: 69-105.

Wilfong R F, Bigner D D, Self D J, Wechsler W. Brain tumor types induced by the Schmidt-Ruppin strain of Rous sarcoma virus in inbred Fischer rats. Acta Neuropathol. 1973;25: 196-206.

We claim:

1. A method of radiosensitizing cancer cells and tumors comprising the steps of
contacting said cancer cells or said tumors with a vector encoding a form of thymidine kinase (TK), wherein said form of TK displays increased nucleoside analog phosphorylation activity when compared to wild type herpes simplex virus thymidine kinase (HSV-TK), and
administering a prodrug to said cancer cells or to said tumors.

2. The method of claim 1 wherein said cancer cells are glioma cells.

3. The method of claim 1 wherein said vector is an adenoviral vector.

4. The method of claim 3 wherein said adenoviral vector is Ad-CMV-TK75.

5. The method of claim 1 wherein said form of TK is a mutant of HSV-TK.

6. The method of claim 5 wherein said mutant of HSV-TK is HSV-TK75.

7. The method of claim 1 wherein said prodrug is acyclovir.

8. A method for killing cancer cells, comprising
contacting said cancer cells with a vector encoding a form of TK, wherein said form of TK displays enhanced activity when compared to wild type HSV-TK,
administering a prodrug to said cancer cells, and
delivering a dose of radiation to said cancer cells wherein said dose is sufficient to kill said cancer cells.

9. The method of claim 8 wherein said cancer cells are glioma cells.

10. The method of claim 8 wherein said vector is an adenoviral vector.

11. The method of claim 10 wherein said adenoviral vector is Ad-CMV-TK75.

12. The method of claim 8 wherein said form of TK is a mutant of HSV-TK.

13. The method of claim 12 wherein said mutant of HSV-TK is HSV-TK75.

14. The method of claim 8 wherein said prodrug is acyclovir.

15. A method of treating cancer in a mammal in need thereof, comprising
delivering to said mammal an effective quantity of a vector encoding a form of TK, wherein said form of TK displays increased activity when compared to wild type HSV-TK,
administering to said mammal an effective quantity of a prodrug, and
providing said mammal with radiation therapy.

16. The method of claim 15 wherein said cancer is glioma.

17. The method of claim 15 wherein said vector is an adenoviral vector.

18. The method of claim 17 wherein said adenoviral vector is Ad-CMV-TK75.

19. The method of claim 15 wherein said form of TK is a mutant of HSV-TK.

20. The method of claim 19 wherein said mutant of HSV-TK is HSV-TK75.

21. The method of claim 15 wherein said prodrug is acyclovir.
